(19) United States
(12) Patent Application Publication
(10) Pub. No.: US 2006/0121034 A1
(43) Pub. Date: Jun. 8, 2006

(54) Treatment for alzheimer's disease and related conditions

(76) Inventor: Jose Luis Castro Pineiro, Bishops Stortford (GB)

Correspondence Address: MERCK AND CO., INC, P O BOX 2000, RAHWAY, NJ, 07065-0907, US

(21) Appl. No.: 10/560,092

(22) PCT Filed: Jun. 4, 2004
(86) PCT No.: PCT/GB04/02381

§ 371 Date: Dec. 9, 2005

(30) Foreign Application Priority Data
Jun. 13, 2003 (GB) 0313772.6

(51) Int. Cl. A61K 39/395; A61K 38/22; A61K 31/192; A61K 31/4747; A61K 31/165

(52) U.S. Cl. 424/145.1; 514/12; 514/618; 514/568; 514/278

(57) ABSTRACT

There is disclosed the combination of a growth hormone secretagogue and at least one agent which modifies the production or processing of Aβ in the brain, said at least one agent being selected from: (a) compounds which inhibit the secretion of Aβ; (b) compounds which selectively inhibit the secretion of the 1-42 isoform of Aβ; (c) compounds which inhibit the aggregation of Aβ; and (d) antibodies which selectively bind to Aβ; for use in treatment or prevention of a disease associated with deposition of Aβ in the brain.

This invention relates to the use of methods and materials for therapeutic treatment of the human body. In particular, it provides methods of treating diseases associated with the deposition of β-amyloid in the brain, such as Alzheimer's disease, or of preventing or delaying the onset of dementia associated with such diseases.

Alzheimer's disease (AD) is the most prevalent form of dementia Its diagnosis is described in the Diagnostic and Statistical Manual of Mental Disorders, 4^{th} ed., published by the American Psychiatric Association (DSM-IV). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase. Variability in the site of the proteolysis mediated by γ-secretase results in Aβ of varying chain length, e.g. Aβ(1-38), Aβ(1-40) and Aβ(142). N-terminal truncations such as Aβ(4-42) are also found in the brain, possibly as a result of variability in the site of proteolysis mediated by β-secretase. For the sake of convenience, expressions such as “Aβ(140)” and “Aβ(1-42)” as used herein are inclusive of such N-terminal truncated variants. After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD.

Other dementing conditions associated with deposition of Aβ in the brain include cerebral amyloid angiopathy, multi-infarct dementia, dementia pugilistica and Down syndrome.

Various interventions in the plaque-forming process have been proposed as therapeutic treatments for AD (see, for example, Hardy and Selkoe, Science, 297 (2002), 353-6). One such method of treatment that has been proposed is that of blocking or attenuating the production of Aβ, for example by inhibition of β- or γ-secretase. Compounds which inhibit γ-secretase are disclosed in WO 01/53255, WO 01/66564, WO 01/70677, WO 01/90084, WO 01/77144, WO 02/30912, WO 02/36555, WO 02/081435, WO 02/081433, WO 03/018543, WO 03/013506, WO 03/013527, WO 03/014075, WO 03/093252, WO 2004/031137, WO 2004/031138, WO 2004/031139, WO 2004/039800 and WO 2004/039370. Compounds which inhibit β-secretase are disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820. Other compounds which inhibit the formation or release of Aβ include those disclosed in WO 98/28268, WO 02/47671, WO 99/67221, WO 01/34639, WO 01/34571, WO 00/07995, WO 00/38618, WO 01/92235, WO 0177086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621.

It has also been reported that inhibition of glycogen synthase kinase-3 (GSK-3), in particular inhibition of GSK-3α, can block the production of Aβ (see Phiel et al, Nature, 423 (2003), 435-9).

Another such method of treatment that has been proposed is that of modulation of the action of γ-secretase so as to selectively attenuate the production of Aβ (142). This results in preferential secretion of the shorter chain isoforms of Aβ, which are believed to have a reduced propensity for self-aggregation and plaque formation, and hence are more easily cleared from the brain, and/or are less neurotoxic. Compounds showing this effect include certain non-steroidal antiinflammatory drugs (NSAIDs) and their analogues (see WO 01/78721 and US 2002/0128319). Compounds which modulate the activity of PPARα and/or PPARδ are also reported to have the effect of lowering Aβ 1-42 (WO 02/100836). NSAID derivatives capable of releasing nitric oxide have been reported to show improved anti-neuroinflammatory effects and/or to reduce intracerebral Aβ deposition in animal models (WO 02/092072; Jantzen et al, J. Neuroscience, 22 (2002), 226-54).

Another such method of treatment that has been proposed is that of administering a compound which blocks the aggregation of Aβ. Compounds having this property include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation include the compounds disclosed in WO 96/28471, WO 98/08868 and WO 00/052048, including the compound known as Apan™ (Praecis), WO 00/064420, WO 03/017994, WO 99/59571 and the compound known as Alzhemed™ (Neurochem), WO 00/149281 and the compositions known as PTI-777 and PTI-00703 (ProteoTech), WO 96/39834, WO 01/83425, WO 01/55093, WO 00/76988, WO 00/76987, WO 00/76969, WO 00/76489, WO 97/26919, WO 97/16194, and WO 97/16191.

Another such method of treatment that has been proposed is that of administering an antibody which selectively binds to Aβ. Such antibodies may be brain-penetrant and capable of binding to insoluble Aβ, as described in WO 99/60024 and WO 00/72880 for example. Alternatively, such antibodies may be capable of sequestering soluble Aβ from biological fluids, without necessarily being brain-penetrant. It is believed that in these circumstances the removal of unbound Aβ from the serum increases the relevant concentration gradient between brain and serum, causing an efflux of Aβ from the brain to the serum. This approach is described in WO 03/016466, WO 03/016467, WO 03/015691 and WO 01/62801. The use of antibodies specific to Aβ-derived diffusable ligands (ADDLS) has also been proposed (WO 2004/031400).

Growth hormone has been proposed for use in treatment of AD. Thus, U.S. Pat. No. 4,902,680 advocates the administration of growth hormone to patients in the advanced stages of AD, while WO 00/13650 discloses that increased levels of growth hormone in the brain provide a neuroprotective effect, and in particular can rescue neurons that would otherwise die as a result of an insult such as that associated with a neurodegenerative disease such as AD. The injection of growth hormone into the brain is contemplated.

Growth hormone secretagogues (GHSs) are compounds which, when administered to an animal (such as a human), stimulate or increase the release of endogenous growth hormone in the animal. Their mode of action and clinical utilities are reviewed by Ankersen et al, Drug Discovery Today, 4 (1999), 497-506; Casanueva and Dieguez, TEM, 10 (1999), 30-8; Smith et al, ibid., 10 (1999), 128-35; Betancourt and Smith, J. Anti-Aging Med., 5 (2002), 63-72; and Ghigo et al, ibid., 5 (2002), 345-56, but there is no mention of treating AD or any other neurodegenerative condition. Patents and patent applications disclosing compounds which are GHSs include U.S. Pat. No. 5,767,124, U.S. Pat. No. 5,536,716, WO 94/13696, EP 0615977B, U.S. Pat. No. 5,578,593; WO 01/04119, WO 98/25897, WO 98/10653, WO 97/36873, WO 97/34604, WO 97/15574, WO 97/11697, WO 96/32943, WO 96/13265, WO 96/02530, WO 95/34311, WO 95/14666, WO 95/13069, WO 94/19367, WO 94/05634 and WO 92/16524 (all assigned to Merck & Co., Inc.); EP 1002802A, EP 0995748A, WO 98/58948, WO 98/58947 and WO 97/24369 (all assigned to Pfizer Inc.); WO 01/34593, WO 00/26252, WO 00/01726, WO 99/64456, WO 99/58501, WO 99/36431, WO 98/58950, WO 98/08492, WO 98/03473, WO 97/40071, WO 97/40023, WO 97/23508, WO 97/00894, WO 96/24587, WO 96/24580, WO 96/22997, WO 95/17423 and WO 95/17422 (all assigned to Novo Nordisk A/S); WO 96/15148 (Genentech Inc.); WO 97/22620 (Deghenghi); WO 02/32888, WO 02/32878, WO 00/49037, WO 00/10565 and WO 99/08699 (all assigned to Eli Lilly and Co.); WO 02/057241 and WO 02/056873 (both assigned to Bayer Corp.); and WO 01/85695, WO 00/54729 and WO 00/24398 (all assigned to Bristol-Myers Squibb Co.). The compounds are recommended for use in promoting the growth of food animals, and in humans for treating physiological or medical conditions characterised by a deficiency in growth hormone secretion, and medical conditions which are improved by the anabolic effects of growth hormone. In some of the above-listed disclosures, the list of treatable conditions includes AD.

The compound disclosed in the aforementioned U.S. Pat. No. 5,767,124 has been the subject of a number of clinical trials in therapeutic fields unrelated to AD (see, for example, Murphy et al, J. Bone Miner. Res., 14, (1999), 1182-8; Chapman et al, J. Clinical Endocrinology and Metabolism, 81, (1996), 4249-57; ibid., 82, (1997), 3455-63; and Svensson et al, ibid., 83, (1998), 362-9).

According to the invention, there is provided the combination of a growth hormone secretagogue and at least one agent which modifies the production or processing of Aβ in the brain, said at least one agent being selected from:

(a) compounds which inhibit the secretion of Aβ;

(b) compounds which selectively inhibit the secretion of the 1-42 isoform of Aβ;

(c) compounds which inhibit the aggregation of Aβ; and

(d) antibodies which selectively bind to Aβ;

for use in treatment or prevention of a disease associated with deposition of Aβ in the brain.

Also according to the invention, there is provided a method of treatment or prevention of a disease associated with deposition of Aβ in the brain comprising administering to a subject in need thereof a therapeutically effective amount of a growth hormone secretagogue (GHS) in combination with a therapeutically effective amount of at least one agent which modifies the production or processing of Aβ in the brain, said at least one agent being selected from:

(a) compounds which inhibit the secretion of Aβ;

(b) compounds which selectively inhibit the secretion of the 1-42 isoform of Aβ;

(c) compounds which inhibit the aggregation of Aβ; and

(d) antibodies which selectively bind to Aβ.

Said disease is typically Alzheimer's disease, cerebral amyloid angiopathy, multi-infarct dementia, dementia pugilistica or Down syndrome, preferably Alzheimer's disease.

The invention further provides a method of treating, preventing or delaying the onset of dementia associated with Alzheimer's disease, cerebral amyloid angiopathy, multi-infarct dementia, dementia puglistica or Down syndrome comprising administering to a patient in need thereof a therapeutically effective amount of a growth hormone secretagogue in combination with a therapeutically effective amount of at least one agent as defined above which modifies the production or processing of Aβ in the brain.

As used herein, the expression “in combination with” requires that therapeutically effective amounts of both a GHS and an agent which modifies the production or processing of AP in the brain (hereinafter termed an “amyloid modifier”) are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the amyloid modifier is an antibody, it will typically be administered parenterally and separately from the GHS.

According to a further aspect of the invention there is provided a pharmaceutical composition comprising, in a pharmaceutically acceptable carrier, a growth hormone secretagogue and an amyloid modifier selected from:

(a) compounds which inhibit the secretion of Aβ;

(b) compounds which selectively inhibit the secretion of the 1-42 isoform of Aβ; and

(c) compounds which inhibit the aggregation of Aβ.

The invention further provides the use, for the manufacture of a medicament for treatment or prevention of a disease associated with deposition of AP in the brain, of a growth hormone secretagogue and an amyloid modifier selected from:

(a) compounds which inhibit the secretion of Aβ;

(b) compounds which selectively inhibit the secretion of the 1-42 isoform of Aβ;

(c) compounds which inhibit the aggregation of Aβ.

Said disease is typically Alzheimer's disease, cerebral amyloid angiopathy, multi-infarct dementia, dementia pugilistica or Down syndrome, preferably Alzheimer's disease.

The GHS and amyloid modifier act synergistically in reducing the accumulation of Aβ in the brain. Therefore, in a further aspect the invention provides a method for retarding, arresting or preventing the accumulation of Aβ in the brain comprising administering to a subject in need thereof a therapeutically effective amount of a growth hormone secretagogue in combination with a therapeutically effective amount of an amyloid modifier as defined above.

Because of the aforementioned synergistic interaction, it is possible to obtain a beneficial therapeutic effect from the administration of doses of the compounds in question that are smaller than would typically be employed for individual administration of the same compounds. For example, a compound which inhibits secretion of Aβ (such as a γ-secretase inhibitor) may be dosed at a level which does not completely suppress the production of Aβ, yet still exert a therapeutic effect comparable to full suppression thereof, as a result of co-administration of the GHS. This has the potential to prevent side-effects that might arise from the suppression of other activities, unconnected with Aβ production, such as the notch signalling process.

Clearance of Aβ from the brain following administration of the relevant compounds may be evidenced by an increase in the level of soluble Aβ in the cerebrospinal fluid and/or the plasma. Alternatively (or additionally), imaging techniques such as magnetic resonance imaging, positron emission tomography, single photon emission computed tomography and multiphoton microscopy may be employed to monitor the extent of Aβ deposition in the brain (see, for example, Bacskai et al., J. Cereb. Blood Flow Metab., 22 (2002), 1035-41).

In one embodiment of the invention, the GHS and amyloid modifier are administered to a patient suffering from AD, cerebral amyloid angiopathy, multi-infarct dementia, dementia pugilistica or Down syndrome, preferably AD.

In an alternative embodiment of the invention, the GHS and amyloid modifier are administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neurol., 56 (1999), 303-8. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the GHS and amyloid modifier are advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, GHS and amyloid modifier are administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42).

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J Psychiatry, 141 (1984), 1356-64).

The invention further provides a kit comprising a first medicament comprising a GHS and a second medicament comprising an amyloid modifier together with instructions for administering said medicaments sequentially or simultaneously to a patient suffering from AD, age-related cognitive decline, MCL cerebral amyloid angiopathy, multi-infarct dementia, dementia pugilistica or Down syndrome.

The GHS used in the invention may be any compound which has the property of stimulating or enhancing secretion of endogenous growth hormone when administered to a subject, for example any of the compounds disclosed in the patents and patent applications listed above. However, preference is given to compounds which are suitable for oral administration.

A first class of GHSs suitable for use in the invention is that disclosed in WO 94/13696, in particular the subset thereof disclosed in EP 0615977B, the disclosure of which is incorporated herein by reference. Preferred examples of GHSs within this class include the compound of formula I: 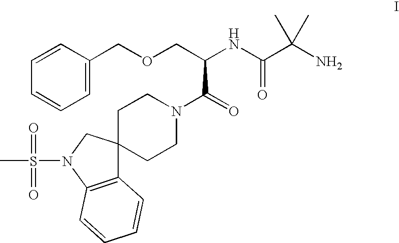
named as N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide, and pharmaceutically acceptable salts thereof, in particular the methanesulfonate salt thereof, which may be prepared as described in U.S. Pat. No. 5,767,124.

A second class of GHSs suitable for use in the invention is that disclosed in U.S. Pat. No. 5,578,593, the disclosure of which is incorporated herein by reference. Preferred example of GHSs within this class include the compound of formula II: 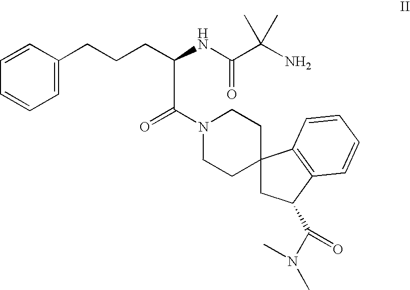
and pharmaceutically acceptable salts thereof, which may be prepared as described in U.S. Pat. No. 5,578,593.

A third class of GHSs suitable for use in the invention is that disclosed in WO 92/16524, the disclosure of which is incorporated herein by reference. Preferred example of GHSs within this class include the compounds of formulae III and IV: 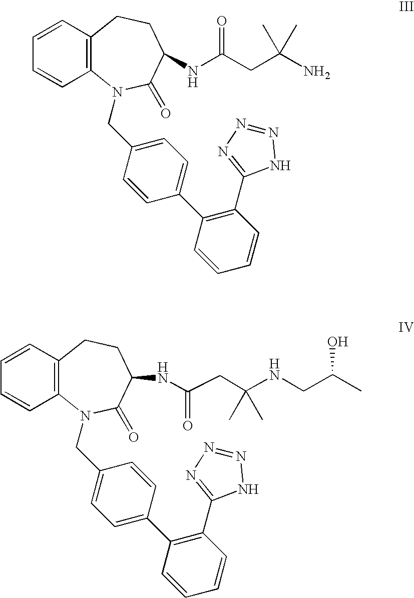
and pharmaceutically acceptable salts thereof, in particular the trifluoroacetate salts thereof, which may be prepared as described in WO 92/16524.

A fourth class of GHSs suitable for use in the invention is that disclosed in WO 97/23508, the disclosure of which is incorporated herein by reference. Preferred examples of GHSs within this class include the compound of formula V, also known as NN703: 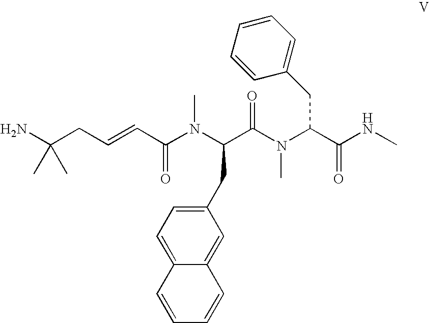
and pharmaceutically acceptable salts thereof, which may be prepared as described in WO 99/64456.

A fifth class of GHSs suitable for use in the invention is that disclosed in WO 97/24369, the disclosure of which is incorporated herein by reference. Preferred examples of GHSs within this class include the compound of formula VI: 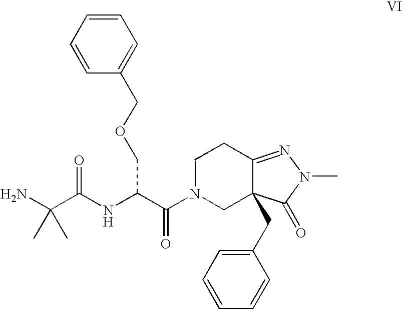
named as 2-amino-N-[2-(3a-(R)-benzyl-2-methyl-3-oxo-2,3,3a,4,6,7-hexahydro-pyrazolo[4,3-c]pyridin-5-yl)-1-(R)-benzyloxymethyl-2-oxo-ethyl]-isobutyramide, and pharmaceutically acceptable salts thereof, in particular the L-tartrate salt, also known as capromorelin, which maybe prepared as described in WO 97/24369 and in Carpino et al, Bioorg. Med. Chem., 11 (2003), 581-90.

A sixth class of GHSs suitable for use in the invention is that disclosed in WO 98/58947, the disclosure of which is incorporated herein by reference. Preferred examples of GHSs within this class include the compound of formula VII: 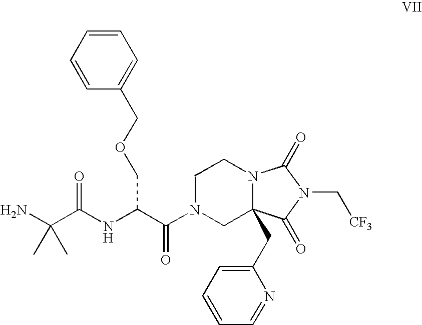
and pharmaceutically acceptable salts thereof, which may be prepared as described in WO 98/58947.

A seventh class of GHSs suitable for use in the invention is that disclosed in WO 99/08699, the disclosure of which is incorporated herein by reference. Preferred examples of GHSs within this class include the compound of formula VIII: 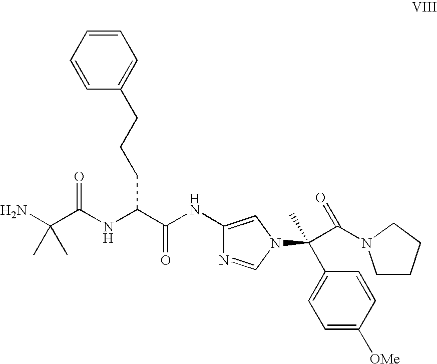
and pharmaceutically acceptable salts thereof, which may be prepared as described in WO 99/08699 and WO 02/32878.

Further GHSs suitable for use in the invention include the compound of formula IX; 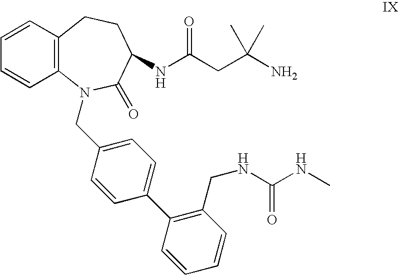
and pharmaceutically acceptable salts thereof, which may be prepared as described in De Vita et al, J. Med. Chem., 41 (1998), 1716-28, and the compound of formula X: 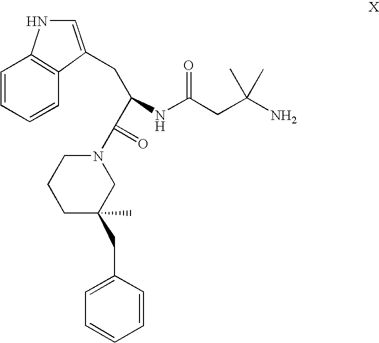
and pharmaceutically acceptable salts thereof, which may be prepared as described in Yang et al, J. Med. Chem., 41 (1998), 2439-41.

Preferably, the GHS is selected from the compounds of formulae I, II, V, VI, VII and IX depicted above, and their pharmaceutically acceptable salts. Most preferably, the GHS used in the invention is the methanesulfonate salt of the compound of formula I which is in one of the polymorphic forms described in U.S. Pat. No. 5,767,124.

In one embodiment of the invention, the amyloid modifier is a compound which inhibits the secretion of Aβ, for example an inhibitor of of γ-secretase (such as those disclosed in WO 01/53255, WO 01/66564, WO 01/70677, WO 01/90084, WO 01/7144, WO 02/30912, WO 02/36555, WO 02/081435, WO 02/081433, WO 03/018543, WO 03/013506, WO 03/013527, WO 03/014075, WO 03/093252, WO 2004/03437, WO 2004/031138 and WO 2004/031139, WO 2004/039800 and WO 2004/039370), or a β-secretase inhibitor (such as those disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820), or any other compound which inhibits the formation or release of Aβ including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621, and also including GSK-3 inhibitors, particularly GSK-3α inhibitors, such as lithium, as disclosed in Phiel et al, Nature, 423 (2003), 435-9.

Within this embodiment, the amyloid modifier is advantageously a γ-secretase inhibitor, preferred examples of which include a compound of formula XI: 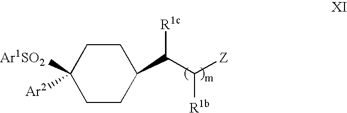
wherein:
m is 0 or 1;
Z represents halogen, CN, NO₂, N₃, CF₃, OR^{2a}, N(R^{2a})₂, CO₂R^{2a}, OCOR^{2a}, COR^{2a}, CON(R^{2a})₂, OCON(R^{2a})₂, CONR^{2a}(OR^{2a}), CON(R^{2a})N(R^{2a})₂, CONHC(═NOH)R^{2a}, heterocyclyl, phenyl or heteroaryl, said heterocyclyl, phenyl or heteroaryl bearing 0-3 substituents selected from halogen, CN, NO₂, CF₃, OR^{2a}, N(R^{2a})₂, COR^{2a}, COR^{2a}, CON(R^{2a})₂ and C₁₋₄alkyl;
R^{1b} represents H, C₁₋₄alkyl or OH;
R^{1c} represents H or C₁₋₄alkyl;
with the proviso that when m is 1, R^{1b} and R^{1c} do not both represent C₁₋₄alkyl;
Ar¹ represents C₆₋₁₀aryl or heteroaryl, either of which bears 0-3 substituents independently selected from halogen, CN, NO₂, CF₃, OH, OCF₃, C₁₋₄alkoxy or C₁₋₄alkyl which optionally bears a substituent selected from halogen, CN, NO₂, CF₃, OH and C₁₋₄alkoxy;
Ar² represents C₆₋₁₀aryl or heteroaryl, either of which bears 0-3 substituents independently selected from halogen, CN, NO₂, CF₃, OH, OCF₃, C₁₋₄alkoxy or C₁₋₄alkyl which optionally bears a substituent selected from halogen, CN, NO₂, CF₃, OH and C₁₋₄alkoxy;
R^{2a} represents H, C₁₋₆alkyl, C₃₋₆cycloalkyl, C₃₋₆cycloalkylC₁₋₆alkyl, C₂₋₆alkenyl, any of which optionally bears a substituent selected from halogen, CN, NO₂, CF₃, OR^{2b}, CO₂R^{2b}, N(R^{2b})₂, CON(R^{2b})₂, Ar and COAr; or R^{2a} represents Ar; or two R^{2a} groups together with a nitrogen atom to which they are mutually attached may complete an N-heterocyclyl group bearing 0-4 substituents independently selected from ═O, ═S, halogen, C₁₋₄alkyl, CN, NO₂, CF₃, OH, C₁₋₄alkoxy, C₁₋₄alkoxycarbonyl, CO₂H, amino, C₁₋₄alkylamino, di(C₁₋₄alkyl)amino, carbamoyl, Ar and COAr;
R^{2b} represents H, C₁₋₆alkyl, C₃₋₆cycloallyl, C₃₋₆cycloalkylC₁₋₆allyl, C₂₋₆alkenyl, any of which optionally bears a substituent selected from halogen, CN, NO₂, CF₃, OH, C₁₋₄alkoxy, C₁₋₄alkoxycarbonyl, CO₂H, amino, C₁₋₄allylamino, di(C₁₋₄akyl)amino, carbamoyl, Ar and COAr; or R^{2b} represents Ar; or two R^{2b} groups together with a nitrogen atom to which they are mutually attached may complete an N-heterocyclyl group bearing 0-4 substituents independently selected from ═O, ═S, halogen, C₁₋₄alkyl, CN, NO₂, CF₃, OH, C₁₋₄alkoxy, C₁₋₄alkoxycarbonyl, CO₂H, amino, C₁₋₄alkylamino, di(C₁₋₄alkyl)amino, carbamoyl, Ar and COAr;
Ar represents phenyl or heteroaryl bearing 0-3 substituents selected from halogen, C₁₋₄alkyl, CN, NO₂, CF₃, OH, C₁₋₄alkoxy, C₁₋₄alkoxycabonyl, amino, C₁₋₄alkylamino, di(C₁₋₄alkyl)amino, carbamoyl, C₁₋₄allylcarbamoyl and di(C₁₋₄alkyl)carbamoyl;
“heterocyclyl” at every occurrence thereof means a cyclic or polycyclic system of up to 10 ring atoms selected from C, N, O and S, wherein none of the constituent rings is aromatic and wherein at least one ring atom is other than C; and
“heteroaryl” at every occurrence thereof means a cyclic or polycyclic system of up to 10 ring atoms selected from C, N, O and S, wherein at least one of the constituent rings is aromatic and wherein at least one ring atom of said aromatic ring is other than C;
or a pharmaceutically acceptable salt thereof.

Such compounds may be prepared as described in WO 03/018543. Preferred examples include those defined by formula XIa: 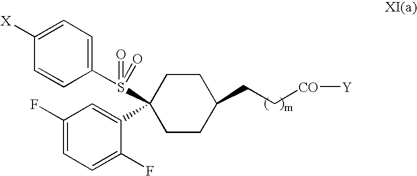
and the pharmaceutically acceptable salts thereof, wherein m is 0 or 1, X is Cl or CF₃, and Y is OH, OC₁₋₆alkyl, NH₂ or NHC₁₋₆alkyl. Particular examples include those in which m is 1 and Y is OH (or the sodium salts thereof), and those in which m is 0 and Y is NH₂ or NHC₁₋₆alkyl.

Another preferred class of γ-secretase inhibitors for use in this embodiment of the invention is that defined by formula XII: 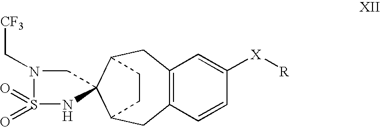
wherein X is a bivalent pyrazole, imidazole, triazole, oxazole, isoxazole, thiazole, isothiazole, thiadiazole or 1,3,4-oxadiazole residue optionally bearing a hydrocarbon substituent comprising 1-5 carbon atoms which is optionally substituted with up to 3 halogen atoms; and
R is selected from:

(i) CF₃ or a non-aromatic hydrocarbon group of up to 10 carbon atoms, optionally substituted with halogen, CF₃, CHF₂, CN, OH, CO₂H, C₂₋₆acyl, C₁₋₄alkoxy or C₁₋₄alkoxycarbonyl;

(ii) a non-aromatic heterocyclic group comprising up to 7 ring atoms of which up to 3 are chosen from N, O and S and the remainder are carbon, bearing 0-3 substituents independently selected from oxo, halogen, CN, C₁₋₆alkyl, OH, CF₃, CHF₂, CH₂F, C₂₋₆acyl, CO₂H, C₁₋₄alkoxy and C₁₋₄alkoxycarbonyl;

(iii) phenyl or 6-membered heteroaryl, either of which bears 0-3 substituents independently selected from halogen, CF₃, CHF₂, CH₂F, NO₂, CN, OCF₃, C₁₋₆alkyl and C₁₋₆alkoxy; and

(iv) N(R^{a})₂ where each R^{a} independently represents H or C₁₋₆alkyl which is optionally substituted with halogen, CF₃, CHF₂, CN, OH, CO₂H, C₂₆acyl, C₁₋₄alkoxy or C₁₋₄alkoxycarbonyl;

or a pharmaceutically acceptable salt thereof.

X is very aptly 5-substituted-thiazol-2-yl, 5-substituted-4-methylthiazol-2-yl, 5-substituted-1-methylpyrazol-3-yl, 1-substituted-imidazol-4-yl or 1-substituted-1,2,4-triazol-3-yl. Preferably, R represents optionally-substituted phenyl or heteroaryl such as phenyl, monohalophenyl, dihalophenyl, trihalophenyl, cyanophenyl, methylphenyl, methoxyphenyl, trifluoromethylphenyl, trifluoromethylphenyl, pyridyl, monohalopyridyl and trifluoromethylpyridyl, wherein “halo” refers to fluoro or chloro. Particularly preferred identities of R—X— include 5-(4-fluorophenyl)-1-methylpyrazol-3-yl, 5-(4-chlorophenyl)-1-methylpyrazol-3-yl and 1-(4-fluorophenyl)imidazol-4-yl. Such compounds may be prepared by methods disclosed in WO 03/093252.

Another preferred class of γ-secretase inhibitors for use in this embodiment of the invention is that defined by formula XIII: 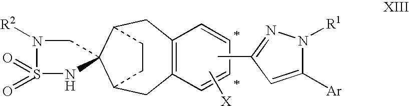
wherein the pyrazole group is attached at one of the positions indicated by an asterisk and X is attached at a position adjacent thereto;
X represents H, OH, C₁₋₄alkoxy, Cl or F;
Ar represents phenyl or 6-membered heteroaryl, either of which bears 0-3 substituents independently selected from halogen, CF₃, CHF₂, CH₂F, NO₂, CN, OCF₃, C₁₋₆alkyl and C₁₋₆alkoxy;
R¹ represents a hydrocarbon group of 1-5 carbon atoms which is optionally substituted with up to 3 halogen atoms; and
R² represents H or a hydrocarbon group of 1-10 carbon atoms which is optionally substituted with up to 3 halogen atoms;
provided that when X is H, R² does not represent 2,2,2-trifluoroethyl;
   or a pharmaceutically acceptable salt thereof.

Preferred examples of compounds of formula XIII include those in which Ar is 4-fluorophenyl, R¹ is methyl, X is H and R² is benzyl, n-propyl, 2,2-dimethylpropyl, n-butyl, isopropyl, t-butyl, 3,3,3-trifluoropropyl, allyl, cyclobutyl or cyclopropylmethyl. Such compounds may be prepared by methods disclosed in WO 2004/039800.

Another preferred class of γ-secretase inhibitors for use in this embodiment of the invention is that defined by formula XIV: 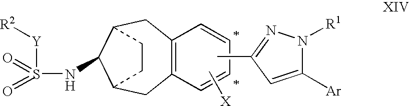
wherein the pyrazole group is attached at one of the positions indicated by an asterisk and X is attached at a position adjacent thereto;
X represents H, OH, C₁₋₄alkoxy, Cl or F;
Y represents a bond, O or NR³;
Ar represents phenyl or 6-membered heteroaryl, either of which bears 0-3 substituents independently selected from halogen, CF₃, CHF₂, CH₂F, NO₂, CN, OCF₃, C₁₋₆alkyl and C₁₋₆alkoxy;
R¹ represents a hydrocarbon group of 1-5 carbon atoms which is optionally substituted with up to 3 halogen atoms; and
R² represents a hydrocarbon group of 1-10 carbon atoms which is optionally substituted with up to 3 halogen atoms, or heteroaryl of 5 or 6 ring atoms optionally bearing up to 3 substituents independently selected from halogen, CF3, CHF₂, CH₂F, NO₂, CN, OCF₃, C₁₋₆alkyl and C₁₋₆alkoxy; or when Y represents NR³, R² and R³ together may complete a heterocyclic ring of up to 6 members which optionally bears up to 3 substituents independently selected from halogen, CF₃, CH2, CH₂F, NO₂, CN, OCF₃, C₁₋₆alkyl and C₁₋₆alkoxy;
R³ represents H or C₁₋₄alkyl, or together with R² completes a heterocyclic ring as defined above;
   or a pharmaceutically acceptable salt thereof.

Preferred examples of compounds of formula XIV include those in which Ar is 4-fluorophenyl, R¹ is methyl, X is H, and either Y is a bond and R² is n-butyl, 4-fluorophenyl, 5-chloro-2-thienyl, 5-isothiazolyl, 6-chloropyridin-3-yl or 2-thienyl, or Y is NR³ and NR²R³ is cyclobutylamino, 2,2,2-trifluoroethylamino, n-propylamino, N-methyl-n-propylamino, dimethylamino, pyrrolidin-1-yl or 4-(trifluoromethyl)piperidin-1-yl. Such compounds maybe prepared as described in WO 2004/039370.

Another preferred class of γ-secretase inhibitors for use in this embodiment of the invention is that defined by formula XV: 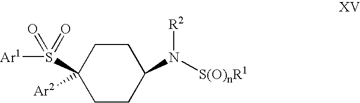
wherein n is 1 or 2;
R¹ represents CF₃ or C₁₋₆alkyl, C₂₋₆alkenyl, C₃₋₉cycloalkyl or C₃₋₆cycloalkylC₁₋₆alkyl, any of which may bear up to 2 substituents selected from halogen, CN, CF₃, OR³, COR³, CO₂R³, OCOR⁴, SO₂R⁴, N(R⁵)₂, and CON(⁵)₂,
or R¹ represents aryl, arylC₁₋₆allyl, C-heterocyclyl or C-heterocyclylC₁₋₆alyl;
R² represents H or C₁₋₄alkyl;
R³ represents H, C₁₋₄alkyl, phenyl or heteroaryl;
R⁴ represents C₁₋₄alkyl, phenyl or heteroaryl;
R⁵ represents H or C₁₋₄alkyl, or two R⁵ groups together with a nitrogen atom to which they are mutually attached complete an azetidine, pyrrolidine, piperidine, morpholine, thiomorpholine or thiomorpholine-1,1-dioxide ring;
Ar¹ and Ar² independently represent phenyl or heteroaryl, either of which bears 0-3 substituents independently selected from halogen, CN, NO₂, CF₃, CHF₂, OH, OCF₃, CHO, CH═NOH, C₁₋₄alkoxy, C₁₋₄alkoxycarbonyl, C₂₋₆acyl, C₂₋₆alkenyl and C₁₋₄alkyl which optionally bears a substituent selected from halogen, CN, NO₂, CF₃, OH and C₁₋₄alkoxy;
“aryl” at every occurrence thereof refers to phenyl or heteroaryl which optionally bear up to 3 substituents selected from halogen, CN, NO₂, CF₃, OCF₃, OR³, COR³, CO₂R³, OCOR⁴, N(⁵)₂, CON(⁵)₂ and optionally-substituted C₁₋₆allyl, C₁₋₆alkoxy, C₂₋₆alkenyl or C₂₋₆alkenyloxy wherein the substituent is selected from halogen, CN, CF₃, phenyl, OR³, CO₂R³, OCOR⁴, N(R⁵)₂ and CON(F⁵)₂; and
“C-heterocyclyl” and “N-heterocyclyl” at every occurrence thereof refer respectively to a heterocyclic ring system bonded through carbon or nitrogen, said ring system being non-aromatic and comprising up to 10 atoms, at least one of which is O, N or S, and optionally bearing up to 3 substituents selected from oxo, halogen, CN, NO₂, CF₃, OCF₃, OR³, COR³, CO₂R³, OCOR⁴, OSO₂R⁴, N(R⁵)₂, CON(R⁵)₂ and optionally-substituted phenyl, C₁₋₆alkyl, C₁₋₆alkoxy, C₂₋₆alkenyl or C₂₋₆alkenyloxy wherein the substituent is selected from halogen, CN, CF₃, OR³, CO₂R³, OCOR⁴, N(R⁵)₂ and CON(R⁵)₂;
or a pharmaceutically acceptable salt thereof.

Preferred examples of compounds of formula XV include those in which R¹ is CF₃, Ar² is 2,5-difluorophenyl and Ar¹ is 4-chlorophenyl, 4-trifluoromethylphenyl or 6-trifluoromethylpyridin-3-yl. Compounds of formula XV may be prepared as described in WO 2004/031139.

In a second embodiment of the invention, the amyloid modifier is a compound which selectively inhibits secretion of the 1-42 isoform of Aβ. Suitable examples of such compounds include the non-steroidal antiinflammatory drugs (NSAIDs) and their analogues disclosed in WO 01/78721 and US 2002/0128319, such as sulindac sulfide, flufenamic acid, ibuprofen, flurbiprofen, fenoprofen, mefenamic acid, indomethacin and (R)-flurbiprofen. A preferred example is (R)-flurbiprofen. Alternatively, an NSAID derivative capable of releasing nitric oxide may be employed (e.g. compounds as disclosed in WO 02/092072 and in Jantzen et al, J. Neuroscience, 22 (2002), 226-54). Preferred examples of NO-releasing compounds include the 4-nitrooxybutyl ester of flurbiprofen (made by NiCox and also known as HCT-1026) and the compound: 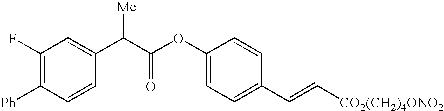
known as NCX-2216 (NiCox). As a further alternative within this embodiment, a compound which modulates the activity of PPARα and/or PPARδ (as disclosed in WO 02/100836) maybe employed.

In a third embodiment of the invention, the amyloid modifier is a compound which inhibits the aggregation of Aβ. Suitable examples include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation suitable for use in the invention include the compounds disclosed in WO 96/28471, WO 98/08868 and WO 00/052048, including the compound known as Apan™ (Praecis); WO 00/064420, WO 03/017994, WO 99/59571 and the compound known as Alzhemed™ (Neurochem); WO 00/149281 and the compositions known as PTI-777 and PTI-00703 (ProteoTech); WO 96/39834, WO 01/83425, WO 01/55093, WO 00/76988, WO 00/76987, WO 00/76969, WO 00/76489, WO 97/26919, WO 97/16194, and WO 97/16191.

In a fourth embodiment of the invention, the amyloid modifier is an antibody which binds selectively to Aβ. Said antibody may be polyclonal or monoclonal, but is preferably monoclonal, and is preferably human or humanized. Preferably, the antibody is capable of sequestering soluble Aβ from biological fluids, as described in WO 03/016466, WO 03/016467, WO 03/015691 and WO 01/62801. Suitable antibodies include humanized antibody 266 (described in WO 01/62801) and the modified version thereof described in WO 03/016466. Further suitable antibodies include those specific to ADDLS as described in WO 2004/031400.

In a particular embodiment of the invention, the amyloid modifier is selected from:

(a) compounds which inhibit the secretion of Aβ;

(b) compounds which selectively inhibit the secretion of the 1-42 isoform of Aβ;

(c) compounds which inhibit the aggregation of Aβ.

Depending on whether they are to be administered together or separately, the GHS and amyloid modifier are typically supplied as single or multiple pharmaceutical compositions comprising the active species and a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and poly(ethylene glycol), and other pharmaceutical diluents, e.g. water, to form a homogeneous preformulation composition containing one or both active species, or pharmaceutically acceptable salts thereof. When referring to these preformulation compositions as homogeneous, it is meant that the active species is or are dispersed evenly throughout the composition so that the composition may be readily subdivided into equally effective unit dosage forms such as tablets, pills and capsules. This preformulation composition is then subdivided into unit dosage forms of the type described above, generally containing from 0.01 to about 500 mg of the active species. Typical unit dosage forms contain from 0.05 to 100 mg, for example 0.05, 0.1, 0.5, 1, 2, 5, 10, 25, 50 or 100 mg, of the active species. Tablets or pills of the pharmaceutical composition(s) can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the pharmaceutical compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) and gelatin.

Pharmaceutical compositions suitable for oral administration are preferred, except when the amyloid modifier is an antibody, in which case parenteral administration of the antibody is preferred, e.g. by subcutaneous, intravenous or intraperitoneal injection.

For treatment or prevention of AD, the GHS and amyloid modifier may be dosed at the levels which are effective for the original purposes of the separate compounds. Thus, the GHS will typically be dosed at levels known to provide increased secretion of endogenous growth hormone in a human subject, and the amyloid modifier at levels known to cause significant inhibition of the secretion of Aβ, or of the 1-42 isoform thereof, or significant inhibition of the aggregation of Aβ, or significant sequestration of soluble Aβ, as appropriate. In many cases, these dosage levels are available from the published literature, but otherwise are readily determined by standard clinical methods. However, as explained above, it may be possible and advantageous to use a smaller dose of the amyloid modifier than would otherwise be indicated, in view of the synergistic interaction with the GHS.

The frequency of dosing of the relevant compounds (e.g. once, twice, three times or four times per day) may be selected according to the pharmacokinetic profiles of the compounds concerned.

In the case of the preferred GHS of formula I, doses of about 0.01 to 5.0 mg/kg per day, preferably about 0.05 to 2.5 mg/kg per day, more preferably about 0.1 to 1.0 mg/kg of body weight per day, may be contemplated. In particular, a dose equivalent to 5 mg, 10 mg or 25 mg of the free base may be administered orally once daily to a patient.

In the case of a compound which inhibits the secretion of Aβ, the dosage may be adjusted so as to provide complete suppression of the secretion of Aβ, or only partial suppression thereof, for example a 50% reduction in Aβ secretion. In the case of a γ-secretase inhibitor of formula XI or XII above, daily oral doses of about 25 to 500 mg per person are contemplated, in particular about 25 to 250 mg per person.

In a further aspect, the invention provides a pharmaceutical composition comprising, in a pharmaceutically acceptable carrier, a compound of formula I or a pharmaceutically acceptable salt thereof and a compound of formula XI(a) or a pharmaceutically acceptable salt thereof. Preferably the compound of formula I is in the form of the methanesulfonate salt. Preferably, the pharmaceutical composition is in a unit dose form suitable for oral administration, such as a tablet or a capsule. In a particular embodiment, said unit dose form contains the equivalent of 5, 10 or 25 mg of the free base of formula I and the equivalent of from 25 to 250 mg of the compound of formula XI(a).

1-13. (canceled)

14. A method of treatment or prevention of a disease associated with deposition of Aβ in the brain comprising administering to a patient in need thereof a therapeutically effective amount of a growth hormone secretagogue in combination with a therapeutically effective amount of at least one agent which modifies the production or processing of Aβ in the brain, said agent being selected from:
(a) compounds which inhibit the secretion of Aβ;
(b) compounds which selectively inhibit the secretion of the 1-42 isoform of Aβ;
(c) compounds which inhibit the aggregation of Aβ; and
(d) antibodies which selectively bind to Aβ.

15. The method of claim 14 wherein the disease is Alzheimer's disease.

16. The method of claim 15 wherein the patient suffers from mild cognitive impairment.

17. The method of claim 16 wherein the patient additionally possesses one or more risk factors for developing Alzheimer's disease selected from:
a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42).

18. The method of claim 14 wherein the growth hormone secretagogue is N-[1(R)-[(1,2-dihydro-1-methanesulfonylspiro[3H-indole-3,4′-piperidin]-1′-yl)carbonyl]-2-(phenylmethyloxy)ethyl]-2-amino-2-methylpropanamide, or pharmaceutically acceptable salt thereof.

19. The method of claim 14 wherein the amyloid modifier is a γ-secretase inhibitor.

20. The method of claim 19 wherein the γ-secretase inhibitor is a compound of formula XIa: 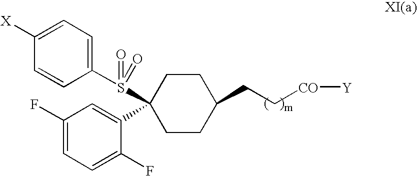
and the pharmaceutically acceptable salts thereof, wherein m is 0 or 1, X is Cl or CF₃, and Y is OH, OC₁₋₆alkyl, NH₂ or NHC₁₋₆alkyl.

21. The method of claim 14 wherein the amyloid modifier is a compound which selectively inhibits the secretion of the 1-42 isoform of Aβ.

22. The method of claim 21 wherein the amyloid modifier is R-flurbiprofen.

23. A pharmaceutical composition comprising in a pharmaceutically acceptable carrier, a growth hormone secretagogue and an amyloid modifier selected from:
(a) compounds which inhibit the secretion of Aβ;
(b) compounds which selectively inhibit the secretion of the 1-42 isoform of Aβ; and
(c) compounds which inhibit the aggregation of Aβ.
